(19) United States
(12) Patent Application Publication
(10) Pub. No.: US 2005/0121033 A1
(43) Pub. Date: Jun. 9, 2005

(54) Respiratory monitoring during gas delivery

(75) Inventors: Eric W. Starr, Allison Park, PA (US); Bernie F. Hete, Kittanning, PA (US)

Correspondence Address: MICHAEL W. HAAS, INTELLECTUAL PROPERTY COUNSEL;RESPIRONICS, INC., 1010 MURRY RIDGE LANE, MURRYSVILLE, PA, 15668, US

(73) Assignee: RIC Investments, LLC., Wilmington, DE (US)

(21) Appl. No.: 11/033,333

(22) Filed: Jan. 11, 2005

Related U.S. Application Data
(63) Continuation-in-part of application No. 10/372,381, filed on Feb. 21, 2003, now Pat. No. 6,849,049.
Application No. 10/372,381 is a continuation of application No. 09/961,618, filed on Sep. 24, 2001, now Pat. No. 6,544,192.
Application No. 09/961,618 is a continuation-in-part of application No. 09/438,081, filed on Nov. 10, 1999, now Pat. No. 6,342,040.
Application No. 09/438,081 is a continuation of application No. 09/030,221, filed on Feb. 25, 1998, now Pat. No. 6,017,315.
(60) Provisional application No. 60/536,132, filed on Jan. 13, 2004.

(51) Int. Cl. A62B 7/00; A61M 16/00

(52) U.S. Cl. 128/204.18; 128/204.23

(57) ABSTRACT

A method and apparatus for monitoring a patient's respiratory status during the delivery of gases, such as supplemental oxygen. In one embodiment, a conduit carries a continuous flow of gas to an airway of a patient over a plurality of respiratory cycles and a gas flow characteristic of the gas in the conduit is monitored using a pressure sensor, a flow sensor, or both. The gas flow characteristic is used to determine a respiratory variable for the patient.

### CROSS-REFERENCE TO RELATED APPLICATIONS

This application claims priority under 35 U.S.C. § 119(e) from provisional U.S. patent application No. 60/536,132 filed Jan. 13, 2004, the contents of which are incorporated herein by reference. This application also claims priority under 35 U.S.C. § 120 as a Continuation-In-Part (CIP) from U.S. patent application Ser. No. 10/372,381 filed Feb. 21, 2003, which is a Continuation of Ser. No. 09/961,618 filed Sep. 24, 2001, now U.S. Pat. No. 6,544,192, which is a continuation-In-Part of Ser. No. 09/438,081 filed Nov. 10, 1999, now U.S. Pat. No. 6,342,040, which is a Continuation of Ser. No. 09/030,221 filed Feb. 25, 1998, now U.S. Pat. No. 6,017,315, the contents of all of these patents and application are hereby incorporated herein by reference.

### BACKGROUND OF THE INVENTION

1. Field of the Invention

The present invention pertains to a method and apparatus for monitoring a patient's respiratory status during the continuous delivery of a gas, such as supplemental oxygen.

2. Description of the Related Art

Supplemental oxygen is widely used for the long-term treatment of chronically ill patients suffering from various respiratory diseases, such as COPD and emphysema. In emergency situations, supplemental oxygen is administered on a short-term basis to relieve acute symptoms, such as shortness of breath and lowered oxygen saturation. Supplemental oxygen is also commonly administered throughout the hospital setting, such as in the operating room during surgery, after surgery in post-op, and in the intensive care units to critically ill patients. In addition, supplemental oxygen is provided to patients on a short-term basis outside the hospital, for example during surgical procedures performed outside the hospital in a doctor's office, dentist's office, surgicenter, or any other location where such procedures are performed.

Conventional practices for administering supplemental oxygen to a patient include fitting a nasal cannula or an oxygen mask on the patient. A conventional nasal cannula typically consists of single lumen tubing with a pair of stubs provided along the length of the tube. The stubs are sized and configured to be situated within the nostrils of the patient, and each stub includes a port through which oxygen flows. The nasal cannula provides more freedom of movement for the patient than other methods of interfacing a flow of supplemental oxygen to a patient, but drawbacks of using the nasal cannula are well known and include unknown delivered FiO₂, irritation of the nose, and potential dislodgment of the cannula from the patient's nostrils. Oxygen masks are simple, inexpensive to use, not subject to easy dislodgment, and reliably administer oxygen levels of 40-60% O₂ to the patient. Oxygen masks designs vary based upon the intended use of the particular mask, but typically include a body that is sized to seat over the nose and mouth of the patient. Oxygen is introduced to an interior of the mask via a single lumen through an oxygen inlet defined in the mask. Expiratory gases are typically vented from the mask through apertures defined in the sides of the mask.

During the administration of oxygen, whether via nasal cannula or mask, it is often desirable to monitor the patient's respiratory status. For example, it is desirable to monitor timing related parameters, such as the patient's breath rate, inspiratory and expiratory times, pressure related parameters, such as the end expiratory pressure, and volume related parameters, such as inspiratory and expiratory tidal volume. Additionally, it is desirable to recognize abnormal breathing patterns, such as Cheyne-Stokes breathing, cessation of breathing (apnea), a reduction in the flow during breathing (hypopnea) or disconnection of the patient from the supplemental oxygen, while the patient is receiving supplemental oxygen. It is particularly desirable to monitor a patient's respiration during the administration of supplemental oxygen while the patent is being given anesthesia, sedative, and/or painkiller, which can occur across the spectrum of care, including at the physician's office, at a surgicenter, in a dentist/orthodontist office, and at a hospital ward.

Existing approaches to measuring the aforementioned parameters include placing sensing elements, such as thermistors, directly in the airflow path of the subject so that the gas flowing into or out of the patient flows across the sensing element. See, e.g., U.S. Pat. Nos. 5,190,048 and 5,413,111 both to Wilkinson. It is also known to place a single lumen at the patient's airway to sense the pressure variations related to the subject's breathing. See, e.g., U.S. Pat. Nos. 5,535,739 and 6,165,133 both to Rapoport. However, these conventional single lumen pressure sensing systems cannot be used when supplemental oxygen is to be provided to the patient because they are unable to provide a clear indication of the pressure variations produced by the patient.

Dual lumen cannula have been developed. A conventional dual lumen cannula includes a first lumen for delivering the supplemental gas to the patient and a second lumen for sampling the exhaled carbon dioxide (CO₂). The second lumen is connected to a vacuum pump that draws a continuous sample of the exhaled breath to the CO₂ monitor. Both lumens are contained in a common conduit housing. See, e.g. U.S. Pat. No. 5,335,656 to Bowe et al. and U.S. Pat. No. 4,989,599 to Carter. Given that these conventional approaches require additional sensing devices at the patient, such as a thermister, and/or a more complicated measurement technique, such as drawing a sample with a vacuum pump, it is desirable to measure these parameters without the associated expense and complexity of the aforementioned approaches.

In addition, conventional dual lumen monitoring systems are used either to monitor the patient's exhaled CO₂ levels, or are used in oxygen conserving devices (OCDs) that pulse or dose the oxygen delivered to the patient. There are instances, however, where it is desirable to provide a continuous flow of oxygen to a patient over multiple breaths, i.e., without pulsing or dosing the oxygen, while monitoring the patient. It is also preferable to provide a system that is less costly and more robust than CO₂ monitoring systems.

### SUMMARY OF THE INVENTION

Accordingly, it is an object of the present invention to provide a simple system to monitor a subject's breathing while the subject is receiving a supplemental gas, such as supplemental oxygen or a gas mixture, that overcomes the shortcomings of conventional monitoring techniques. This object is achieved according to the present invention by providing a respiratory therapy and monitoring apparatus that includes a first conduit having a proximal end, a distal end, and a first lumen defined, therethrough from the proximal end to the distal end. A continuous flow of gas is delivered from a gas supply to an airway of a patient over a plurality of respiratory cycles. A first sensor is operatively coupled to the airway of the patient while the continuous flow of gas is passing through the conduit. The first sensor monitors a gas flow characteristic indicative of pressure or flow in the first conduit. In addition, a processor is provided that is adapted to determine at least one respiratory variable of such a patient based on the gas flow characteristic.

In a further embodiment of the present invention, the offset or bias present in the gas flow characteristic due to continuous introduction of the supplemental gas is removed. This is accomplished by programming executed by the processor, dedicated hardware, or both. In this manner, the present invention provides a system that both delivers a continuous flow of supplemental gas to a patient and that allows the patient to be monitored by a simple and reliable pressure or flow sensor, so that the condition of the patient can be evaluated in real-time during the delivery of supplemental gas.

It is yet another object of the present invention to provide a respiratory therapy and monitoring method that does not suffer from the disadvantages associated with conventional measurement techniques. This object is achieved by providing a method that includes (1) providing a first conduit, (2) delivering, via the first conduit, a continuous flow of gas from a gas supply to an airway of a patient over a plurality of respiratory cycles, (3) monitoring a gas flow characteristic using a pressure sensor, a flow sensor, or both operatively coupled to the airway of the patient while the continuous flow of gas is passing through the conduit, and (4) determining at least one respiratory variable of such a patient based on the gas flow characteristic. In a further embodiment of the present invention, the offset or bias present in the gas flow characteristic due to continuous introduction of the supplemental gas is removed, by the processor, dedicated hardware, or a combination of the two.

It is a further object of the present invention to provide a method of displaying a respiratory characteristic of a patient. This object is achieved by displaying a time varying respiratory characteristic over at least a portion of a patient's respiratory cycle during a current (n) respiratory cycle and a time varying respiratory characteristic over at least a portion of such a patient's respiratory cycle during a prior respiratory cycle. These time varying respiratory characteristics during at least two respiratory cycles are displayed in a superimposed fashion.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of “a”, “an”, and “the” include plural referents unless the context clearly dictates otherwise.

### BRIEF DESCRIPTION OF THE DRAWINGS

FIG. 1 is a schematic diagram of a first embodiment of a monitoring and therapy delivery system according to the principles of the present invention;

FIG. 2 is a schematic diagram of the system of FIG. 1 shown in use on a patient;

FIGS. 3-5 are schematic diagrams illustrating three alternative techniques for accounting for an offset in a measured gas flow characteristic caused by introducing a continuous flow of gas to the airway of the patient while monitoring the patient;

FIG. 6A is a graph of a representative pressure signal measured by the pressure sensor of the monitoring and therapy delivery system of FIG. 1, and FIG. 6B is a graph showing the patient's breathing patterns and offset bias flow signal separated from the measured pressure signal;

FIG. 7 is a schematic diagram of a second embodiment of a monitoring and therapy delivery system according to the principles of the present invention;

FIG. 8 is a graph illustrating a patient's breathing frequency recorded over a period of time using the monitoring and therapy delivery system of the present invention;

FIG. 9 is a schematic diagram of a third embodiment of a monitoring and therapy delivery system according to the principles of the present invention;

FIG. 10 is a detailed view of the distal end of the conduit in the monitoring and therapy delivery system of FIG. 9;

FIG. 11 is a schematic diagram of a fourth embodiment of a monitoring and therapy delivery system according to the principles of the present invention;

FIG. 12 is a schematic diagram of a fifth embodiment of a monitoring and therapy delivery system according to the principles of the present invention;

FIG. 13 is a schematic diagram of a sixth embodiment of a monitoring and therapy delivery system according to the principles of the present invention;

FIG. 14 is a schematic diagram of a seventh embodiment of a monitoring and therapy delivery system according to the principles of the present invention;

FIGS. 15 and 16 are perspective and front views, respectively, of a cannula with filter and a housing suitable for use in the monitoring and therapy delivery system of the present invention;

FIG. 17 illustrates an exemplary waveform display suitable for use in displaying a monitored waveform according to the principles of the present invention;

FIG. 18 is a graph of a hypothetical flow measured by a flow in the monitor of the present invention

FIG. 19 illustrates an eight embodiment of a monitoring and therapy delivery system according to the principles of the present invention;

FIG. 20 illustrates a ninth embodiment of a monitoring and therapy delivery system according to the principles of the present invention; and

FIG. 21A is a rear perspective view, FIG. 21B is a top view, and FIG. 21C is a front perspective view of a tenth embodiment of a monitoring and therapy delivery system according to the principles of the present invention.

### DETAILED DESCRIPTION OF THE PRESENTLY PREFERRED EMBODIMENTS OF THE INVENTION

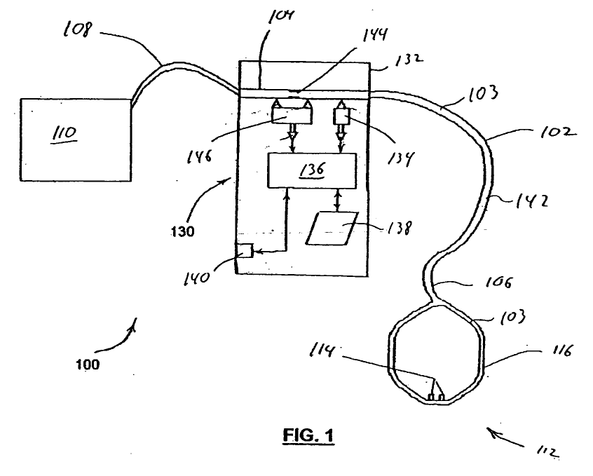

FIG. 1 schematically illustrates an exemplary embodiment of a respiratory monitoring and therapy delivery system 100 according to the principles of the present invention. Monitoring and therapy delivery system 100 includes a conduit or cannula 102 having a proximal end 104 and a distal end 106. The terms “conduit” and “cannula” are used interchangeably. A connector 108 is provided at the proximal end of the conduit to connect the conduit to a supply of gas 110, such as a tank of oxygen with a pressure regulator or an oxygen concentrator. Of course, the present invention contemplates that any type of gas suitable for delivery to a patient can be used as gas supply 110. Conduit 102 includes a lumen 103, i.e., a bore, defined therein to carry a flow of gas between the proximal end and the distal end. In the embodiment illustrated in FIG. 1, conduit 102 includes a single lumen, thereby simplifying the manufacturability and use of the system.

A patient interface device, generally indicated by reference numeral 112, is provided at distal end 106 of conduit 102. In the embodiment illustrated in FIG. 1, the patient interface device is a pair of nasal prongs 114, each of which includes a port for delivering a flow of gas to both nares of a patient. The prongs at the distal end of the conduit do not seal the patient's nostril, so that some gas is permitted to flow from the patient's nose to the ambient atmosphere around the prongs. In this embodiment, the distal end of conduit 102 includes a loop 116 to allow the distal end of the conduit to wrap around a patient's head, as is the case with a standard oxygen tubing.

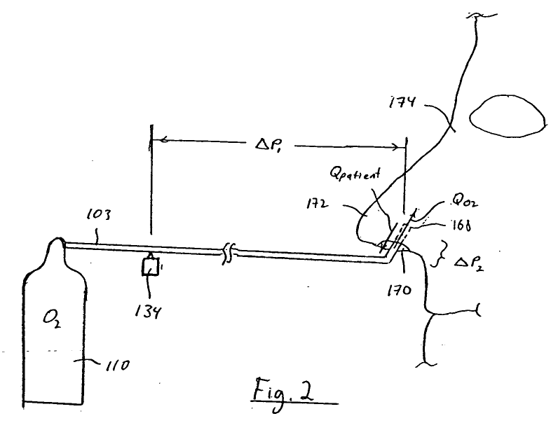

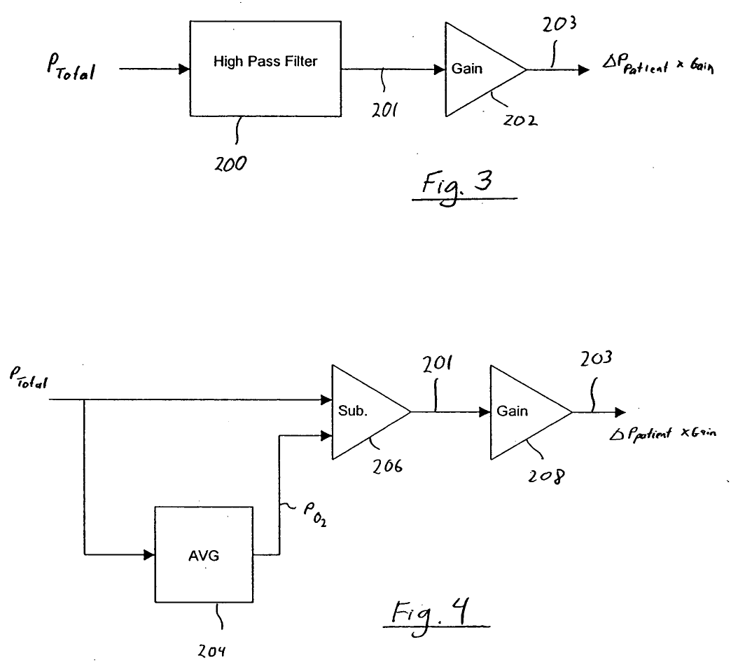

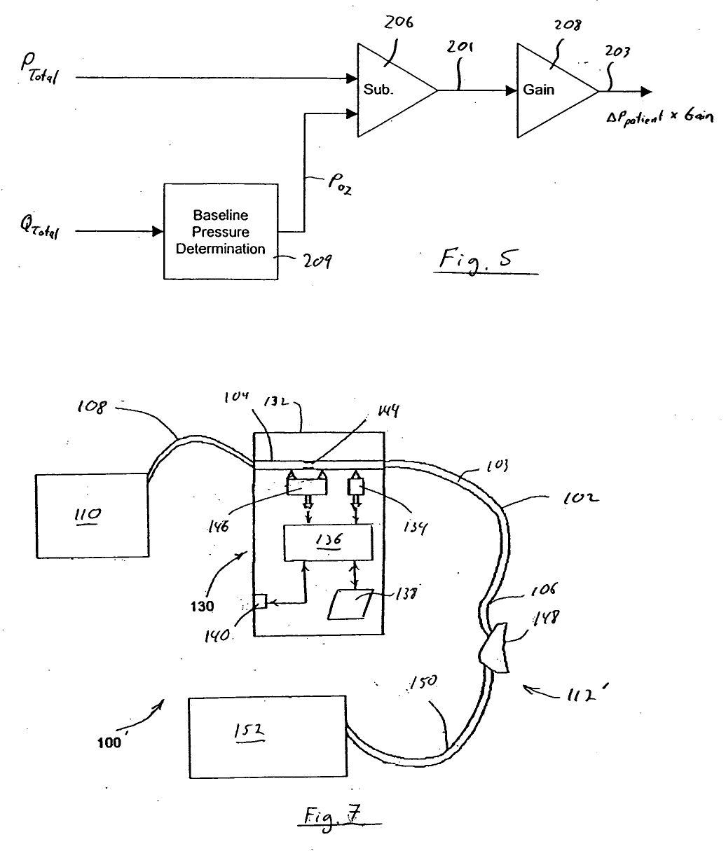

Patient interface device 112 can be any device suitable for coupling the flow of gas from conduit 102 to an airway of a patient. Examples of suitable patient interface devices include a nasal mask, oral mask or mouthpiece, nasal/oral mask, nasal cannula, trachea tube, intubation tube, and hood or full face mask. It is to be understood that this list of suitable interface devices is not intended to be exclusive or exhaustive. For example, in the embodiment illustrated in FIG. 2 and discussed in detail below, the patient interface device is a nasal/oral mask.

Monitoring system 100 includes a respiratory monitoring system, generally indicated by reference numeral 130, that is preferably provided in a housing 132. Respiratory monitoring system 130 includes a sensor 134 operatively coupled to conduit 102. In the present embodiment, sensor 134 is a pressure sensor that monitors a pressure within the conduit and outputs a first pressure signal indicative thereof. More specifically, pressure sensor 134 measures the pressure within the lumen relative to a constant or relatively constant reference pressure, such as absolute or atmospheric pressure. A single pressure sensor port or tap into conduit 102 is provided from communicating the pressure sensor with the lumen in the conduit. This sensing port can have any configuration known in the art, such as a single tap flush to the lumen wall or a single tap protruding into the interior of the lumen.

A processor 136 receives the pressure signal and determines from that signal at least one respiratory variable of the patient, as discussed in greater detail below. Processor 136 is preferably a microprocessor capable of implementing a stored algorithm, which determines from the monitored pressure from pressure sensor 134 the respiratory variable of interest. Of course, processor 136 includes the necessary memory and processing capability to implement the features of the present invention.

The present invention further contemplates that respiratory monitoring system 130 includes an input/output interface 138 for communicating, information, data, and/or instructions and any other communicatable items, collectively referred to as “data”, between a user and processor 136. Examples of common input/output interfaces suitable for this purpose include a keypad and display that visually indicates at least one respiratory variable in a human perceivable format.

The present invention also contemplates providing other communication techniques, either hard-wired or wireless, for communicating with processor 136 from a remote location. For example, a terminal 140 can be provided that enables data to be loaded from a data storage device, such as a disk, CD-ROM, memory card, smart card, etc., into processor 136 or loaded onto the storage device from the processor. Other exemplary, interface devices and techniques adapted for use with the respiratory measurement system include, but are not limited to, an RS-232 port, CD reader/writer, DVD reader/writer, RF link, modem (telephone, cable, or other). In short, any conventional technique for providing, receiving, or exchanging data with processor 136 is contemplated by the present invention as terminal 140.

In the embodiment illustrated in FIG. 1, conduit 102 includes a first portion 142 coupled to housing 132 and patient interface device 112. It is to be understood that the distance from the tap where pressure sensor 134 measures the pressure to the ports at the patient interface should not be too large. Otherwise, the pressure drop across this portion of the conduit may be too great. For example, in one embodiment of the present invention, the length of this portion of the conduit is approximately seven (7) feet. However, conduit lengths up to fifty (50) feet are also contemplated by the present invention.

The embodiment illustrated in FIG. 1 also shows connector 108 provided at proximal end 104 of conduit 102 as being a tube. It is to be understood that the length of this connector can vary. For example, one embodiment of the present invention contemplates connecting the housing directly to gas supply 110, thereby minimizing the distance between the gas source and the location where pressure sensor 134 taps into conduit 102. Still other embodiments of the present invention contemplate making this connector quite long, so long as the pressure drop through connector 108 does not become too large.

Referring now to FIGS. 1-4, 6A and 6B, a description will be provided as to how processor 136 determines, from the pressure measured by the pressure sensor, at least one respiratory variable of the patient. The pressure (P_{Total}) measured by pressure sensor 134 is the summation of a pressure drop ΔP₁ and a pressure drop ΔP₂. Pressure drop ΔP₁ is the pressure drop that occurs along the length of lumen 103 between the location of the pressure sensor and the open end 168 of the lumen inserted into a nare 170 of a nose 172 of a patient 174. Pressure drop ΔP₂ is a pressure drop from open end 168 of conduit 102 to the ambient atmosphere, which is at the opening of the nostril. Thus, P_{Total}=ΔP₁+ΔP₂.

Pressure drop ΔP₁ is determined based on the flow Q_{O2} of the supplement gas through lumen 103 and the resistance to flow R_{Tube} that exists along the length of conduit 102 between the location of the pressure sensor and open end 168. Thus, ΔP₁=Q_{O2}*R_{Tube}. Pressure drop ΔP₂ is based on the pressure drop at the nose due to the bias flow of the supplemental gas (ΔP_{O2}) and the pressure drop at the nose due to the patient's respiration (ΔP_{Patient}). Pressure drop ΔP_{O2} is determined based on the flow Q_{O2} of the supplement gas through lumen 103 and the resistance to flow at the nose R_{Nose}. Thus, ΔP_{O2}=Q_{O2}* R_{Nose}. Pressure drop ΔP_{Patient} is determined based on the patients respiratory flow Qₚₐₜᵢₑₙₜ and the resistance to flow at the nose R_{Nose}. Thus, ΔP_{Patient}=Q_{Patient}*R_{Nose}. Pressure drop ΔP₂ can be expressed as follows:
ΔP₂=ΔP_{O2}+ΔP_{Patient}, or (1)
ΔP₂=Q_{O2}*R_{Nose}+Q_{Patient}*R_{Nose}, or (2)
ΔP₂=(Q_{O2}+Q_{Patient})R_{Nose} (3)

It can thus be appreciated that the pressure (P_{Total}) measured by pressure sensor 134 can be rewritten as:
P_{Total}=Q_{O2}*R_{Tube}+(Q_{O2}+Q_{Patient})R_{Nose} (4)

Because the signal of primary interest in the present application is the pressure resulting from the patient breathing into the patient interface (ΔP_{Patient}), the present invention compensates or removes the pressure drops that are due to the administration of the continuous supplemental flow of gas. That is, the present invention contemplates removing the bias flow Q_{O2} from equation (4).

One embodiment by which the bias flow Q_{O2} is removed from the total pressure P_{Total} is shown in FIG. 3. In this embodiment, the measured (total) pressure P_{Total} is provided to a high pass filter 200. The cutoff frequency of the high pass filter is set such that it is less than the lowest breathing rate that the system would be expected to encounter. For example, the human breathing frequency is typically in a range of 0.1 Hz to 3 Hz. Thus, the cutoff frequency is set to be less than 0.1 Hz. Output 201 of high pass filter 200, which corresponds to ΔP_{Patient}, is provided to an amplifier 202, so that output 203 of amplifier 202 is a signal or waveform that corresponds to ΔP_{Patient} times a Gain.

It should be noted that the present invention contemplates that the function of high pass filter 200, amplifier 202, or both can be implemented in hardware, software, or a combination thereof. It should also be understood that other cutoff frequencies can be used in the high pass filter. For example, the present invention contemplates that the respiratory therapy and monitoring system of the present invention can be used on other species of animals that my have different breathing frequencies. In which case, a breathing frequency appropriate for the species using the device should be selected as the cutoff frequency for the high pass filter.

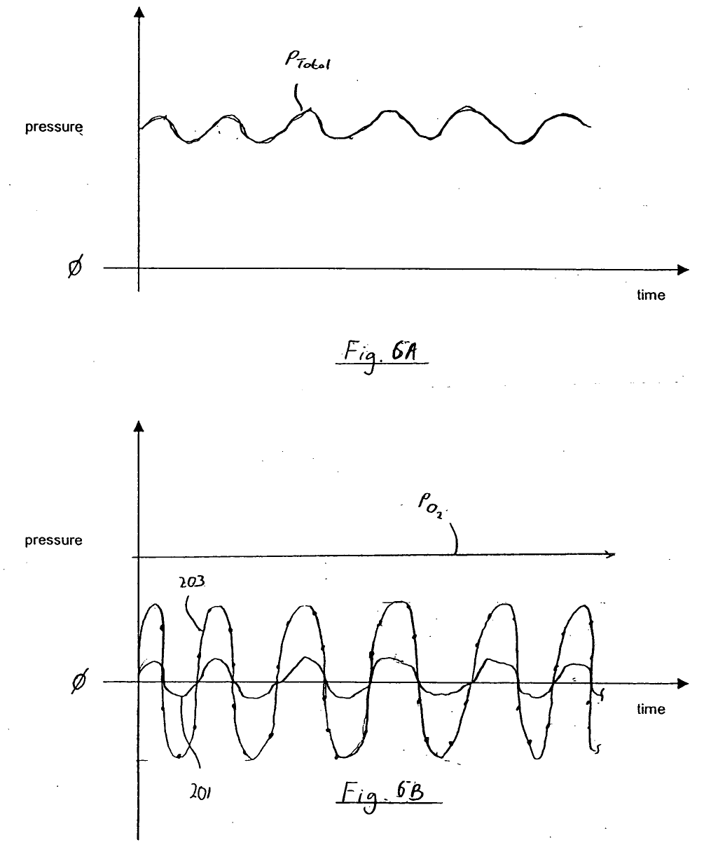

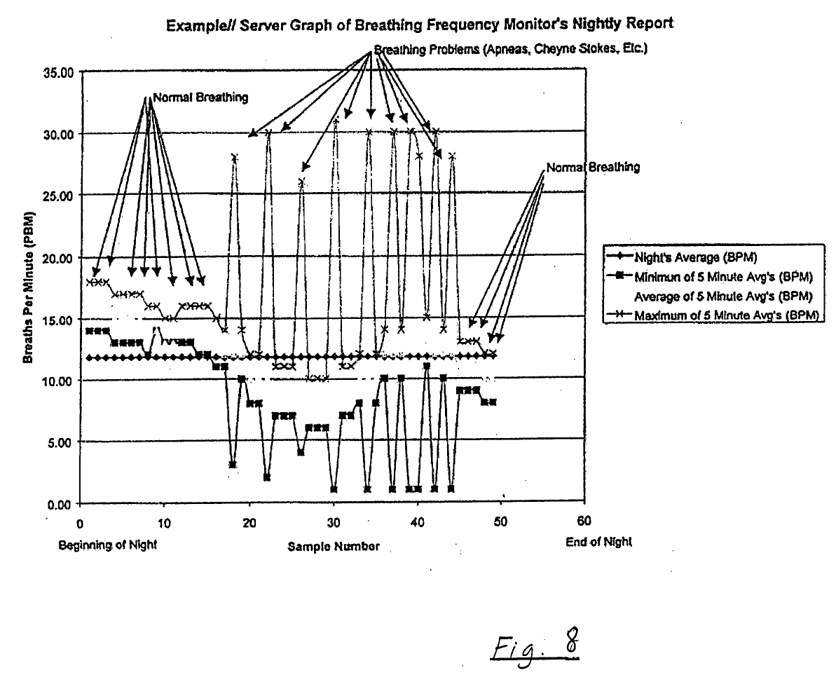

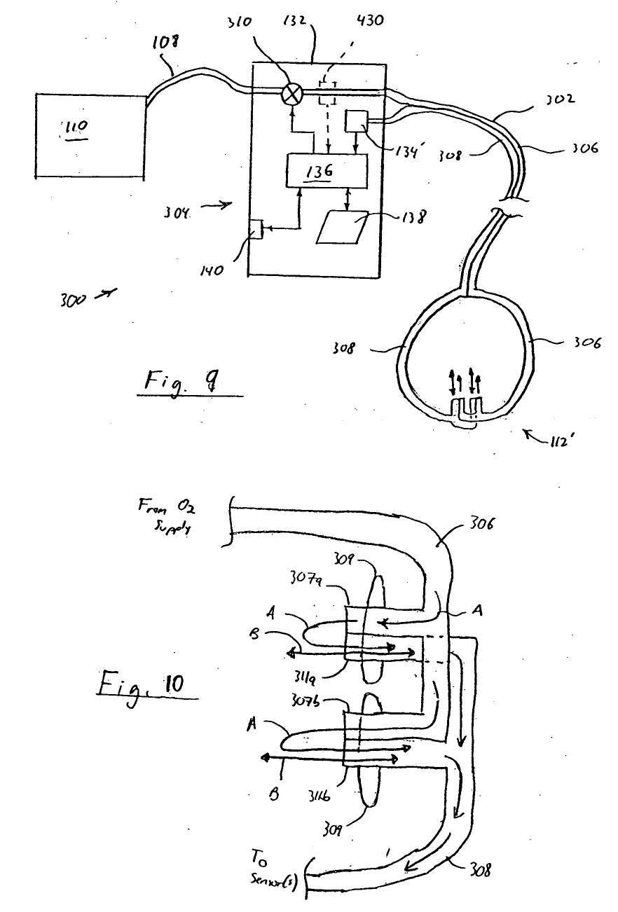

Another embodiment by which the effects of the bias flow Q_{O2} is removed from the total pressure P_{Total} is shown in FIG. 4. In this embodiment, the measured (total) pressure P_{Total} is provided to an averaging device 204, such as a microprocessor or digital signal processor (DSP), that determines the average for the measured pressure signal. The output of averaging device 204 and the measured pressure P_{Total} are provided as input to a subtracting element 206, such as a difference amplifier, such that the output of averaging device 204 is subtracted from P_{Total} yielding a signal or waveform that corresponds to the patient breathing pattern or ΔP_{Patient}. This signal is preferably amplified by an amplifier 208, as needed, to produce a signal having the desired fidelity, i.e., ΔP_{Patient}×Gain, where the Gain is a gain provided by amplifier 208.

As with the previous embodiment, the function of averaging device 204, subtracting element 206, amplifier 208, or any combination thereof can be implemented in hardware, software, or in a combination of thereof. While two exemplary techniques have been described above and shown in FIGS. 3 and 4, it can be appreciated that a variety of other techniques are contemplated by the present invention. Thus, the present invention is not intended to be limited to the specific techniques described herein.

The signal from pressure sensor 134 is not identical to the pressure at the patient interface, i.e., at the airway of the patient, due to the pressure drop ΔP₁ that occurs along the length of the conduit between the patient's airway and the pressure sensor. If the pressure at the patient's airway is desired, the present invention contemplates using the signal from pressure sensor 134 to determine pressure level at the patient interface. This is accomplished by measuring the pressure at a location along conduit 102 via pressure sensor 134 and offsetting this measurement by the known pressure drop associated with the conduit from the location where the pressure measurement is taken, to the distal end of the conduit where the gas exists the conduit. It can be appreciated that accounting for the pressure drop in the conduit ΔP₁ requires determining that pressure drop in advance. In one embodiment of the present invention, the known pressure drop is stored in memory and the stored value is used to determine the pressure at the patient based on the measured pressure after being processed to remove the offset due to the supplemental gas flow.

Another embodiment contemplates accounting for the known pressure drop in the conduit using additional hardware. For example, the present invention contemplates adding a flow sensor 146 along conduit 102 to measure the continuous flow of oxygen delivered to the patient. The signal from flow sensor 146 is provided to control 136, which includes a look-up table or other suitable correlation function relating predetermined pressure drops over a range of flows for the conduit being used. That is, by measuring the gas flow, a look-up table can be used to determine the pressure drop in the conduit ΔP₁ associated with that flow rate. Thus, the pressure at the patient interface can be found by subtracting ΔP₁ from the pressure P_{Total} measured via pressure sensor 134.

A further embodiment of the present invention contemplates storing the known pressure drop for different conduits, i.e., conduits of different length, inside diameter, or both. The specific conduit attached to the monitoring system is then entered or learned by the system using any conventional technique so that the appropriate pressure drop for that conduit can be used in determining the pressure at the patient interface. For example, the present invention contemplates using any conventional connector encoding or identifying technique for automatically indicating to processor 136 that type or size conduit is being used when the conduit is attached to housing 132. The present invention also contemplates calculating the pressure drop ΔP₁ based on the length and the diameter of the conduit, which can be easily determined at the time the system is assembled.

Measurement of the pressure level allows pressure related respiratory variables, such as the inspiratory positive airway pressure (IPAP), expiratory positive airway pressure (EPAP), positive end expiratory pressure (PEEP), and continuous positive airway pressure (CPAP) to be determined. Of course, measuring IPAP, EPAP, and CPAP are only possible when the appropriate pressure support therapy is being provided to the patient in addition the gas flow through conduit 102. The use of a pressure support therapy in combination with the flow of gas through conduit 102 is discussed below with reference to FIG. 7.

A further embodiment of the present invention contemplates adding a flow sensor 146 in respiratory monitoring system 130, so that flow related variables, such as rate of oxygen flow (Q_{O2}) and volume (V_{O2}), can be measured. In one exemplary embodiment, this is achieved by providing a flow restriction 144 in conduit 102 and a flow sensor 146 in the form of a differential pressure sensor that measures the differential pressure across the restriction within the lumen. Restriction 144 may be any restriction or obstruction known in the art for flow measurement, and includes but is not limited to, fixed geometries, such as the venturi, and fixed orifice devices as well as variable geometries, such as variable orifice devices. The differential pressure measured by flow sensor 146 is provided to processor 136, which uses that measurement to determine the rate of flow of gas through conduit 102, the volume of gas over any given period of time, or both using conventional techniques.

It will be readily apparent to one of skill in the art of patient monitoring and/or flow sensing that other techniques for determining the rate of flow of gas in conduit 102 can be used as the flow sensor. Examples of other conventional flow sensing techniques suitable for use with the present invention include, but are not limited to, ultrasonic flow meters, optical flow meters, diverting flow meters, and thermally based flow meters. In addition, the present invention contemplates using flow meter 146 in conjunction with pressure sensor 134 to obtain multiple measurements of gas flow characteristics.

FIG. 5 illustrates an exemplary embodiment of a processing configuration by which the bias due to the flow of supplemental gas delivery pressure is removed from the total flow (Q_{Total}) measured by flow sensor 146 and the pressure measured by pressure sensor 134. In this embodiment, the measured flow Q_{Total} is provided to a baseline pressure determining device 209, such as a microprocessor or digital signal processor (DSP), which determines a baseline pressure ΔP_{O2} from the measured flow Q_{Total}. This is accomplished as discussed above, by providing a look-up table or other suitable correlation function that relates the measured flow to a pressure drop. This look-up table or pressure versus flow relation is determined in advance for the conduit being used in the monitoring system.

As with the embodiment illustrated in FIG. 4, the output of baseline pressure determining device 209 and the measured (total) pressure P_{Total} are provided as input to a subtracting element 206, such as difference amplifier, such that the ΔP_{O2} is subtracted from P_{Total} yielding a signal or waveform that corresponds to the patient breathing pattern or ΔP_{Patient}. This signal is preferably amplified by an amplifier 208 as needed to produce a signal having the desired fidelity.

Because pressure or flow variations from a baseline level are generated as a result of changes in pressure at the patient interface due to patient breathing, time related changes, or fluctuations from the baseline pressure or flow can be analyzed to permit the determination of the transitions between the inspiratory and expiratory phases of breathing. This allows time related respiratory variables, such as breathing frequency, to be determined from the time difference of successive breaths of either the inspiratory or expiratory demarcations. The present invention contemplates using any conventional technique for determining the demarcations or transition between the inspiratory and the expiratory phase of the patient's breathing cycle.

Inspiratory time, expiratory time, and derived indices, such as percentage of inspiratory time of total breath, and the I:E (inspiratory-to-expiratory) ratio can be determined once the inspiratory phase and the expiratory phase are determined. Integration of the time related changes in pressure also permit approximations of the inspiratory and expiratory tidal volumes to be made using signal processing methods known in the art, including but not limited to integral functions.

A second embodiment of a respiratory monitoring system 100′ is shown in FIG. 7. In this embodiment, patient interface device 112′ is a face mask 148 coupled to distal end 106 of conduit 102. Additionally, a patient circuit 150 is connected to patient interface device 112′ (face mask 148) to carry a flow of gas from a ventilator or pressure support system 152 to the patient's airway in addition to the gas flow provided by conduit 102.

Facemask 148, when connected via patient circuit 150 allows the administration of continuous positive airway pressure (CPAP), bi-level positive airway pressure, auto-titrating pressure support, PAPP, PAV, ventilator, or any other conventional pressure support therapy. Patient circuit 150 may consist of a single lumen breathing tube with an exhalation port or valve provided on or near the mask, or two breathing tubes, with one tube used to apply inspiratory flow and the other tube to allow for expiratory flow from the patient. It should be readily apparent to one skilled in the art of pressure and flow sensing that other gases or gas mixtures, such as anesthetic agents or helium/oxygen mixtures, may be used with the present invention requiring only changes to the calibration of the respiratory measurement system.

Although the present invention contemplates that the information gathered via monitoring system 100, 100′ can be output, displayed, or transmitted in any one of a variety of formats, one example of such an output is shown in FIG. 8. This figure is a graph of breathing frequency recorded over a night derived from the present invention. Five minute averages of minimum and maximum breathing frequency may be plotted to easily identify periods of relatively stable breathing patterns from variable breathing patterns.

Breathing pattern analysis includes the identification of abnormal forms of breathing, such as, but not limited to, Cheyne-Stokes breathing, Kussmaul breathing, apnea, hypopnea, and snoring. Breathing pattern analysis may be performed using the respiratory timing variables derived according to the present invention. Cheynes-Stokes breathing is seen with some central nervous system disorders, uremia, and some sleep patterns and is characterized by repeating cycles of waxing and waning in the depth of breathing including a period of apnea. Kussmaul breathing is seen in coma or diabetic ketoacidosis and is characterized by a deep, rapid respiratory pattern. Any conventional technique for determining these breathing patterns can be used in the present invention.

A third embodiment of respiratory therapy and monitoring system 300 according to the principles of the present invention is discussed below with reference to FIGS. 9 and 10. This embodiment is similar in many respects to the respiratory therapy and monitoring system of the first embodiment shown in FIG. 1. However, in this third embodiment, a dual lumen cannula 302 is used to connect gas supply 10 and respiratory monitoring system 304 to the patient, instead of the single lumen cannula of FIG. 1.

Dual lumen cannula 302 includes a first lumen 306 that connects gas supply 110 to the patient and a second lumen 308 that connects a sensor to the patient. In the illustrated embodiment, the sensor is a pressure sensor. It is to be understood, however, that the sensor to which second lumen 308 is connected could also be a flow sensor. The first lumen has a pair of prongs 307a and 307b that insert into the user's nares 309, and the second lumen has a pair of prongs 311a and 311b that also insert into the nares. Gas flows out of ports defined in prongs 307a and 307b into the user's nostrils due to the continuous supply of gas being delivered to the first lumen from the gas source. This gas flow is illustrated by arrows A in FIG. 9. Pressure is applied cyclically to prongs 311a and 311b in second lumen 308 during patient respiration. The proximal end of cannula 302 is connected to a pressure sensor 134′.

It can be appreciated that the flow or pressure of gas in second lumen 308, e.g., the output of pressure sensor 134′, will include a pressure drop ΔP_{O2} due to the flow of supplemental gas being delivered to the patient. For this reason, this third embodiment of the present invention, like the previous embodiments, contemplates removing or compensating for the pressure drop is generated due to the administration of the supplemental flow of gas ΔP_{O2}. The techniques discussed above for accomplishing this function are, therefore, applicable to this embodiment.

It should also be noted that the proximal end of second lumen 308 can be connected to a flow sensor. That is, pressure sensor 134′ can be replaced with a flow sensor, and the proximal end of the second lumen can be open to atmosphere so that a flow is created in the second lumen due to the bias flow of gas. During patient respiration, gas flows into and out of prongs 311a and 311b cyclically. This flow of gas is illustrated by arrows B. In which case, all of the discussion given above regarding measuring pressure and processing the pressure signal is equally applicable to measuring flow and processing the flow signal, including removing or compensating for the pressure drop ΔP_{O2} due to the administration of the supplemental flow of gas Q_{O2}.

Respiratory therapy and monitoring system 300 shown in FIG. 9 also includes a supplemental gas flow control valve 310 associated with first lumen 306. Valve 310 controls the delivery of the supplemental gas to the patient, and corresponds to the flow control valve found in a variety of oxygen delivery systems. For example, this valve is used to set the flow rate in liters-per-minute for the flow of oxygen to the patient. In the illustrated embodiment, valve 310 operates under the control of processor 136. Of course, valve 310 can be a manually actuated or remotely actuated valve. Although not shown, the present invention contemplates providing a similar valve in any of the other embodiments of the present invention.

One skilled in the art can appreciate that any movement of the cannula connecting the patient to the monitoring system is likely to introduce noise in the pressure or flow measurement conducted by the monitoring system. While this noise can be removed using the filtering and/or averaging techniques discussed above with respect to the bias flow removal, i.e., removing the pressure drop ΔP_{O2} component from the P_{Total} signal, another approach that can be used alone or in conjunction with these techniques is shown in FIG. 11.

Respiratory therapy and monitoring system 320 in FIG. 11 is similar to that shown in FIG. 1, except that conduit 322, which is connected to the patient, includes an artifact rejection lumen 324 in addition to single lumen 103. Artifact rejection lumen 324 is preferably physically connected to patient monitoring and gas flow delivery lumen 103 so that any movement in lumen 103 also occurs or is translated into artifact rejection lumen 324. A distal end 326 of artifact rejection lumen 324 is open to ambient atmosphere, but is situated relative to the patient such that any pressure or flow resulting from respiration of the patient or delivery of the supplemental gas is not “detected” by the open distal end portion 326 of the artifact rejection lumen. For example, one embodiment of the present invention contemplates terminating artifact rejection lumen 324 such that the distal end is located at or near the patient's ear, where gas flow due to patient respiration or oxygen delivery is unlikely to be detected. The present invention also contemplates that the distal end of artifact rejection lumen 324 can be closed, i.e., not open to the atmosphere.

A proximal end of artifact rejection lumen 324 is connected to a second sensor 328. Second sensor 328 is preferably the same type of sensor used in the gas monitoring system. Thus, if pressure sensor 138 is used, sensor 328 preferably should also be a pressure sensor. If flow sensor 146 is used, sensor 328 is preferably also a flow sensor. If both pressure sensor 138 and flow sensor 146 are used, sensor 328 should include both a pressure sensor and a flow sensor. The present invention also contemplates mixing the pressure and flow sensors to accomplish this same function. It is not necessary to pair a pressure sensor with a pressure sensor and a flow sensor with a flow sensor.

Movements in lumen 103 appear as noise in the output of pressure sensor 134 and/or flow sensor 146. Because lumen 103 and lumen 324 are physically connected, any movement taking place in lumen 103 also takes place in lumen 324. As a result, any noise due to movement of conduit 322 is detected by sensor 328, which outputs a signal corresponding thereto. This “noise” signal output by sensor 328 is subtracted from the signal output by pressure sensor 134 and/or flow sensor 146, thus minimizing the noise due to tube movement. Subtracting out the noise component of the signal measured by pressure sensor 134 and/or flow sensor 146 based on the signal detected by sensor 328 preferably takes place in processor 136 and is accomplished using any signal processing technique.

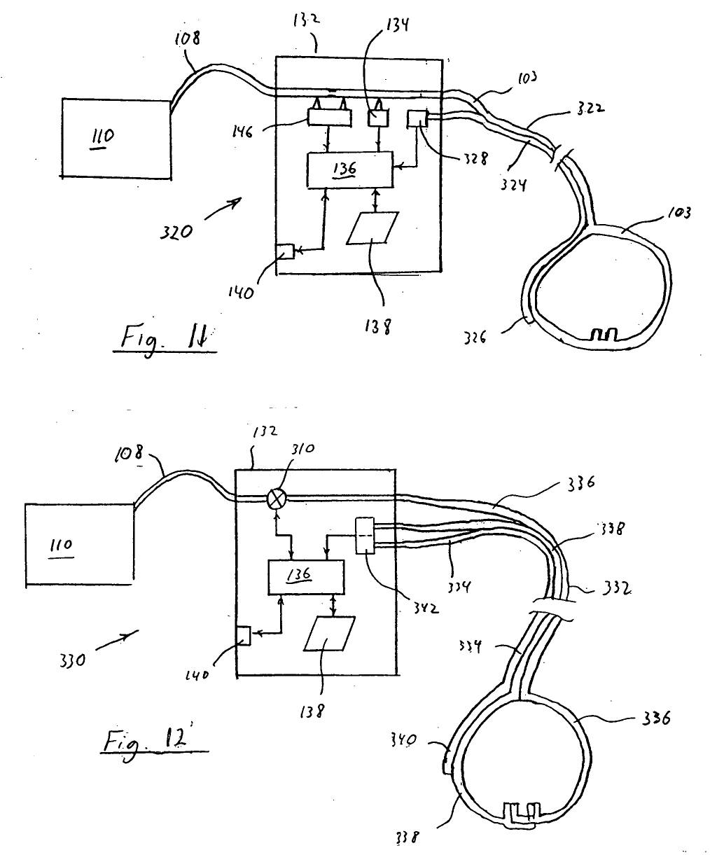

FIG. 12 illustrates another embodiment for removing the noise or artifact resulting from tube movement. Respiratory therapy and monitoring system 330 in FIG. 12 is similar to that shown in FIG. 9, except that conduit 332, which is connected to the patient, includes an artifact rejection lumen 334 in addition to first lumen 336 and second lumen 338. First lumen 336 corresponds to lumens 103 and 302 in that its function is to communicate the supplemental gas flow from the gas source to the airway of the patient. Second lumen 338 and artifact rejection lumen 334 provide the patient monitoring and noise rejection functions.

Artifact rejection lumen 334 is physically connected to first and second lumens 336 and 338, such that any movement of the conduit takes place in all lumens. A distal end 340 of artifact rejection lumen 334 is open to ambient atmosphere and situated relative to the patient such that any pressure or flow resulting from respiration of the patient or delivery of the supplemental gas is not detected by open distal end portion 340, for example, by terminating artifact rejection lumen 334 near the patient's ear, where gas flow due to patient respiration or oxygen delivery is unlikely to be detected by lumen 334.

A proximal end of artifact rejection lumen 334 and a proximal end of second lumen 338 are connected to opposite sides of a differential pressure transducer 342. As a result of this connection across the differential pressure transducer, any noise signal or artifact, i.e., movement of lumens 334 and 338, creates substantially identical pressure variations on each side of the diaphragm in the differential pressure transducer, thereby canceling each other out. Thus, the only remaining pressure sensed by differential pressure transducer 342 is the change in pressure detected by second lumen 338. In essence, the differential pressure transducer performs, via hardware, the noise measurement and cancellation techniques discussed above with respect to FIG. 11. The output of differential pressure transducer 342 is the total patient pressure P_{Total} with any noise due to motion of conduit 332 effectively suppressed or eliminated.

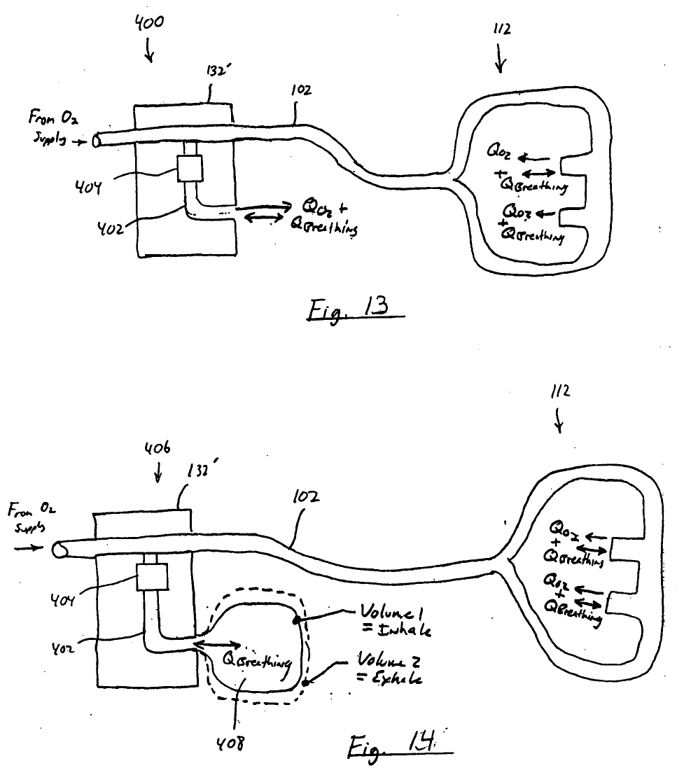

FIG. 13 is a schematic diagram of a sixth embodiment of a monitoring and therapy delivery system 400 according to the principles of the present invention. This embodiment is also similar to that of FIG. 1 except that an exhaust conduit 402 is provided from conduit 102 to ambient atmosphere. A flow sensor 404 is provided to measure the flow of gas passing through exhaust conduit 402. The flow of gas Q_{Total} measured by flow sensor 404 will include (1) a flow of gas Q_{O2} due to the continuous flow of oxygen being introduced into conduit 102, and (2) a flow of gas Q_{Breathing} due to patient breathing. Thus, flow sensor 404 provides a flow measurement comparable to the flow measurement made by flow sensor(s) in the previous embodiments.

FIG. 14 is a schematic diagram of a seventh embodiment of a monitoring and therapy delivery system 406 according to the principles of the present invention. This embodiment is similar to that of FIG. 13, except that a gas receiving reservoir 408 is provided at the end of exhaust conduit 402. Reservoir 408 is closed so that gas passing through flow sensor 404 does not exhaust to atmosphere. Reservoir 408 is also formed from a compliant material, so that its volume can change as the pressure in conduit 102 changes. For example, during inhalation, reservoir 408 has a first volume (Volume 1), and during exhalation, in which the pressure in conduit 102 is greater, the reservoir has a second, greater volume (Volume 2). This expansion of reservoir 408 as the pressure in the conduit increases is illustrated by the dashed line in FIG. 14.

Because reservoir 408 is compliant, it allows gas to flow in exhaust conduit 402 during breathing. However, because the reservoir is closed, there is no continuous flow of gas to atmosphere. Thus, the flow measurement made by flow sensor 404 is directly related, i.e., proportional to, the a flow of gas Q_{Breathing} due to patient breathing.

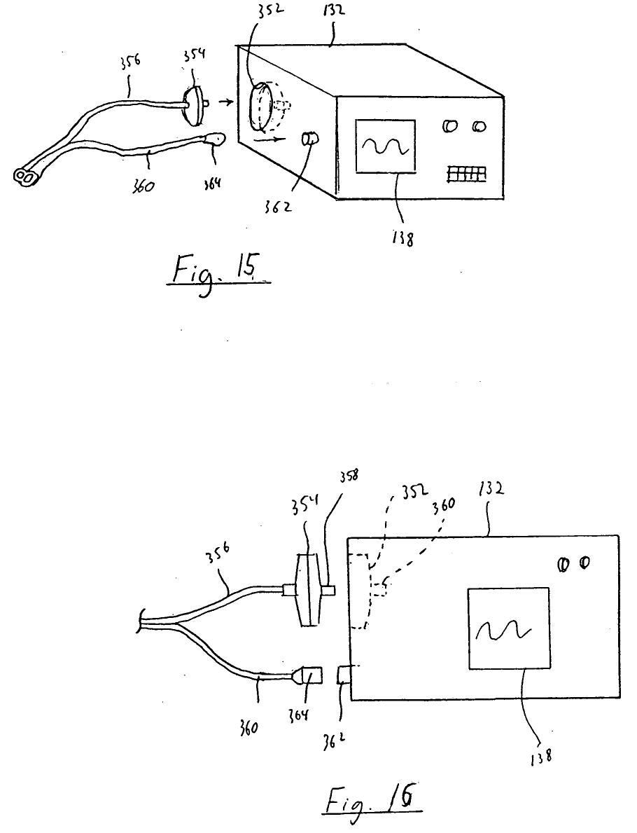

It is common when using a cannula to provide a bacterial filter or similar filtering element in the gas flow path of the cannula. The bacterial filter protects the sensor from contaminants carried in the gas. The present invention contemplates using the bacterial filter, and, more specifically, the bacteria filter housing, to assist in connecting any one of the cannula (lumens) discussed above to housing 132. One example of this concept is illustrated in FIGS. 15 and 16.

Housing 132 includes a receptacle 352 that is sized and configured to receive a housing 354 of a bacteria filter attached to a cannula 356. Receptacle 352 and housing 354 of the bacteria can have any shape imaginable so long as they are complementary, allowing at least a portion of housing 354 to fit within receptacle 352. For example, in the illustrated embodiment, bacteria filter 354 includes an optional stem portion 358 and receptacle 352 includes a stem receptacle 360 into which the stem portion inserts.

This configuration for receptacle 352 and bacteria filter 354 serves two purposes. First, placing all or at least a portion of the bacteria filter within the receptacle protects the bacteria filter from damage, such as from bumping, and streamlines the cannula running from the housing to the patient so that no bulky items are provided on this length of relatively slim, lightweight tubing. Second, this configuration allows the bacteria filter to provide a solid attachment between the cannula and the housing. The relatively large size of the bacteria housing also makes it easy for a user to plug the bacteria filter into the receptacle.

In the embodiment shown in FIGS. 15 and 16, cannula 356 corresponds to cannula 103, 308, 324, 334, or 338 in the above-described embodiments. A second cannula 360 is connected to a supplemental gas port 362 via attachment portion 364. Cannula 360 corresponds to lumen 306 or 336 from the above-described embodiments.

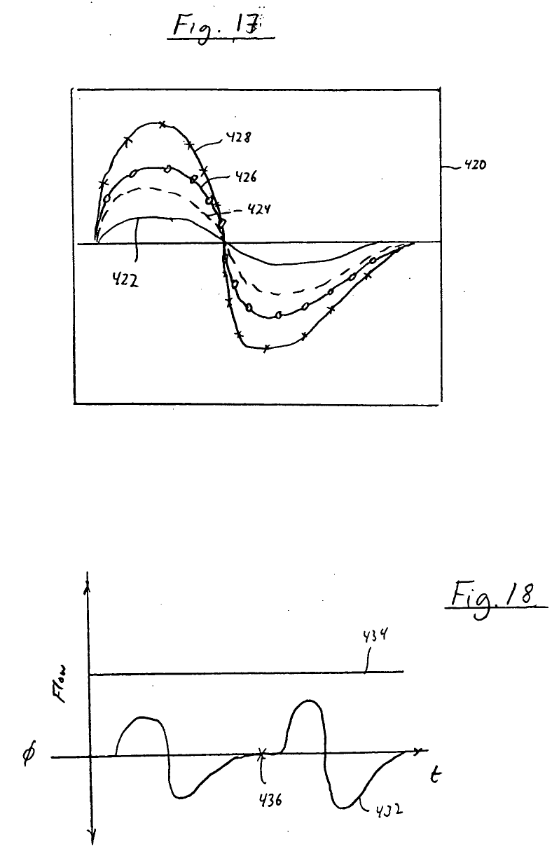

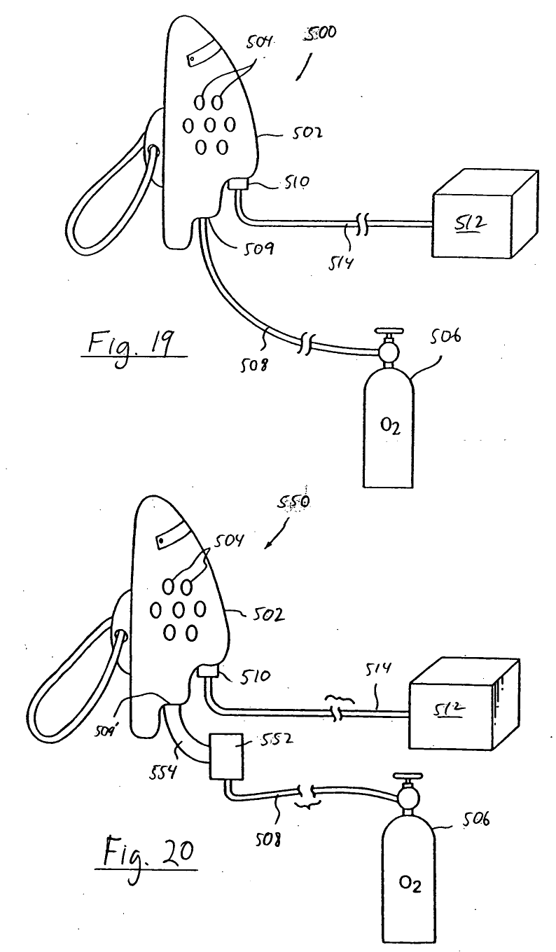

FIG. 17 illustrates an exemplary display 420 suitable for use in displaying a waveform or a plurality of waveforms corresponding to the monitored pressure resulting from the patient breathing into the patient interface. More specifically, the present invention contemplates displaying the ΔP_{Patient} waveforms for the last n breaths in a superimposed fashion, so that the user can quickly visually compare the current waveform with one or more previous waveforms.

For example, waveform 422 shown in display 420 represents the waveform of ΔP_{Patient} for the current respiratory cycle n. Waveform 424 represents the waveform of ΔP_{Patient} for the previous respiratory cycle (n-1). Waveform 426 represents the waveform of ΔP_{Patient} for the respiratory cycle (n-2). Waveform 428 represents the waveform of ΔP_{Patient} for the previous respiratory cycle (n-3). It can be appreciated that this visual representation allows the user to quickly see that, in this hypothetical example, the patient is experiencing an abrupt reduction in breathing, which is indicative, for example of the patient experiencing an apnea. It can also be appreciated that any monitored parameter, such as patient flow or volume can also be displayed using this technique.

Referring back to FIG. 9, a further embodiment of the present invention will now be described. This embodiment contemplates adding a flow sensor 430 to conduit 302 to measure the flow of gas Q_{O2} in conduit 302. By measuring the flow of gas via sensor 430 and the pressure via pressure sensor 134′ the following two important physiological parameters can be determined: (1) the volumetric patient flow in liters per minute (1 pm); and (2) the fractional inspired oxygen concentration (FIO₂) can be determined using this configuration and these measurements.

The volumetric patient flow rate is a quantitative measure of the amount of gas passing into and out of the patient during the respiratory cycle. FIG. 18 is a graph illustrating a hypothetical flow waveform 432 which corresponds to the flow measured by flow sensor 430 with the oxygen flow 434 removed, while a constant flow of supplemental oxygen is being delivered, and while a patient is breathing into patient interface device 112′. Removing the bias due to the supplemental oxygen flow from the output of flow sensor 430 is accomplished, for example, using a high pass filter, or using any of the other techniques discussed herein.

Preferably using software, a point 436 on waveform 432, when the patient flow is substantially zero, can be determined. At this point, all of the pressure inside the nose is caused by the supplemental oxygen flow. The pressure drop (ΔP) is measured by pressure sensor 134′ when the patient flow is zero and while supplemental oxygen is being delivered. In addition, the oxygen flow rate (Q_{O2}) is measured at this time. These measured values, when patient flow is zero, are used to determine the resistance (R) of the system according to equation (1) as follows:
ΔP=R*Q² (5)

Solving this equation for R yields,
R=Q²/ΔP (6)

Thus, the resistance of the system can be determined using Q_{O2} for Q when ΔP is measured corresponding to time 436. This calculated resistance can then be used to determine the quantitative flow Q(t) at all other times by solving equation (1) for flow as follows:
Q(t)=\sqrt{ΔP(t)·R}, (7)
where ΔP(t) is the pressure measured by pressure sensor 134′.

It should be noted that equation (7) may not accurately represent patient flow because the flow at the nose may include turbulent and laminar flow, while equation (7) presumes that the flow will be turbulent. The relationship between pressure and flow for a laminar flow is a first order equation similar to that of equation (5), ΔP=Q*R. In addition, the value of R is determined based on pure, i.e., 100%, oxygen flow. A patient, however, does not inhale and exhale 100% oxygen at the nose using the system of FIG. 9, for example. Thus, the present invention contemplates applying a correction factor to equation (7) to maintain the accuracy of the quantitative flow determination.

The correction factor to apply to the calculated flow value is determined by comparing the volume of gas inhaled to the volume of gas exhaled. It is assumed that over a period of time, e.g., over the last five respiratory cycles, the volume inhaled should equal the volume exhaled. If it does not, it can be assumed that calculated flow Q(t) should be corrected.

The method for compensating for these factors involves using the following expression:
ΔP=R*Q^{(x)}, (8)
where x has a value between 1 (e.g., purely laminar flow) and 2 (e.g., purely turbulent flow). The present invention contemplates adjusting the value for x based on the inspiratory to expiratory volume match. If the inspiratory volume exceeds the expiratory volume, the value of x is decreased toward 1, and vice versa. The amount by which x is changed, how frequently this change is made, and the threshold levels that give rise to a change, can be determined based on predetermined criteria or can be changed based on the monitored condition of the patient.

The present invention also contemplates estimating the volumetric flow rate through a given conduit, such as through the nostril Q_{Patient}. However, to do so requires that the pressure drop from this inside of the nostril to ambient atmosphere ΔP₂ and the resistance of the nostril R_{Nose} be known. See FIG. 2. It can be appreciated that each patient has a uniquely sized nostril. For this reason, the resistance of the nostril is different for each patient.

The present invention contemplates estimating the resistance of the nostril R_{Nose} by estimating the size of the patient's nostril. To this end, the present invention contemplates providing a set of nostril gages, where the resistance associated with each different sized gage in the set of gages is predetermined. The gages are used to determine the gage size that best fits a particular patient. Once the nostril size is gauged, the associated resistance for that gage size is used to estimate the volumetric flow. For example, R_{Nose} in equation (4) is now known (actually estimated) based on the nostril gage. The other variables in this equation are also known except for Q_{Patient}. Equation (4) can be solved for Q_{Patient} to provide a volumetric estimation of the patient flow. It should be noted that equation (4) is a first order equation. Thus, it assumes a laminar flow. For a turbulent flow a second order variation is used as follows:
P_{Total}=Q_{O2}²* R_{Tube}+(Q_{O2}+Q_{Patient})²R_{Nose} (9)

The present invention also contemplates that a combination of equation (4) and (9) representing the presence of both laminar and turbulent flow can be used for the purposes discussed herein, such as solving for Q_{Patient}.

Once the patient flow rates, volumes inhaled (V_{inhaled}), and volumes exhaled (V_{exhaled}) are determined, the FIO₂ can be estimated by calculating the volume of oxygen inhaled (V_{O2inhaled}) and by using equation (10). The volume of oxygen inhaled (V_{O2inhaled}) is determined based on the flow measurement from flow sensor 430.

$${FIO}_{2} = \frac{( ( {V}_{inhaled} - {V}_{O2inhaled} ) ( 0.21 ) ) + ( ( {V}_{O2inhaled} ) ( 1.0 ) )}{{V}_{inhaled}} . \qquad ( 10 )$$

It should be noted that the value “1.0” in equation (10) is used assuming that 100% oxygen is being delivered to the patient. This value can be adjusted to correspond to other delivered oxygen concentrations.

The respiratory therapy and monitoring system of the present invention can be a stand-alone system or it can be combined with other medical devices. When combined with other medical devices or systems, it can be integrated into that system or provided as a module that selectively attaches to the other medical device.

The respiratory therapy and monitoring system of the present invention is particularly well suited for use in situations where a patient is receiving a surgical procedure outside the hospital. In this configuration, the respiratory therapy and monitoring system delivers oxygen to the patient while monitoring his or her respiratory characteristics, such as whether he or she is breathing normally or experiencing an apnea. This occurs before, during, or after the patient receives a medication used during the procedure, such as an anesthetic, i.e., a general anesthetic, a sedative, a pain reliever, or a combination thereof. Until the present invention, there has been little or no patient monitoring in these situations, and deaths have been known to occur. The present invention provides a cost effective monitoring capability in such an environment, so that that the physician or other caregiver can be warned of any deterioration in the patient's condition.

Heretofore, a quantitative measurement of the flow and/or volume of gas entering or exiting the patient is possible by first determining the resistance (R) or the system or the nose (Rₙₒₛₑ). This resistance can then be used to determine the flow/volume by measuring the pressure across the resistance. The present invention, as described above, determines the resistance of the system by solving for resistance when the flow in the system (with bias removed) is zero, i.e., see equation (6) above. The above-described embodiments also teach estimating the resistance of the nose using a sizing gage, where the resistance associated with each different sized gage in the set of gages is predetermined. These resistance determination techniques are used if the resistance of the orifice is not known. The present invention also contemplates providing a patient interface having a flow element with a known resistance. However, in this embodiment, all of the gas passing into or out of the patient must pass through the flow element.

FIG. 19 illustrates an eight embodiment of a monitoring and therapy delivery system 500 according to the principles of the present invention. System 500 includes a patient interface 502 that includes a flow element 504, i.e., an exhaust path to atmosphere, with a known resistance. A gas supply 506 provides a constant supply of breathing gas, such as oxygen or an oxygen mixture, to the interior of the patient interface via a conduit 508 and port 509. Gas supply 506 is any suitable supply, such as an oxygen tank or oxygen concentrator. In the illustrated exemplary embodiment, patient interface 502, which is a mask, includes a second port 510 that communicates a sensor 512 to the interior portion of the mask via a conduit 514. It is to be understood that the breathing gas need not be directly provided to the user interface, as shown in FIG. 19. On the contrary, the gas can be provided to the conduit 514, thereby avoiding the need to provide two ports in the mask.

A plurality of holes are provided in the mask to define the flow element. It is to be understood, however, that any venting system for communicating the interior of the mask with ambient atmosphere, while creating a pressure drop across the flow element, is contemplated by the present invention. Because the resistance of this flow element is known and all gas exists the mask through this flow element, the flow or volume of gas delivered to or from patient can be quantitatively measured. Techniques for making this quantitative flow or volume pressure determination are discussed in detail in U.S. Pat. Nos. 6,544,192; 6,342,040; and 6,017,315, the contents of which are hereby incorporated herein by reference.

A still further embodiment of a monitoring and therapy delivery system 550 is shown in FIG. 20. The patient interface 502 of this embodiment is substantially similar to that shown in FIG. 19, except that a nebulizer or a medication delivery device 552 is provided in addition to the supply of breathing gas or oxygen. This embodiment allows a supply of aerosolized medication or other therapeutic or medicated gas to be delivered to the patient via a relatively large bore conduit 554 connected to patient interface device 502 at port 509′. The present invention contemplates that the internal cross-sectional area of conduit 554 and port 509′ should be larger than that of a standard oxygen cannula to allow the aerosolized medication to be delivered efficiently to the patient.

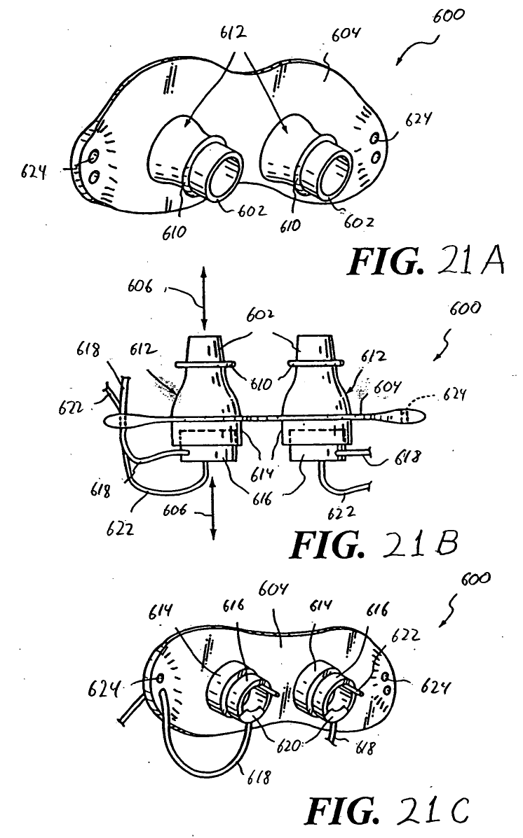

FIGS. 21A-21C illustrate a nasal prong patient interface 600 according to a further embodiment of the present invention suitable for use in the monitoring and therapy delivery system of the present invention. As in the embodiments of FIGS. 19 and 20, patient interface 600 is configured to capture all of the flow of gas to and from the user so that the flow/volume can be quantatively measured. Nasal prong patient interface 600 includes a pair of protruding portions 602 supported by a base member 604. In an exemplary embodiment of the present invention, protruding portions 602 are integral with base member 604 and all are formed from a relatively flexible and biocompatible material. Protruding portions 602 insert into the patient's nares and seal the nares so that all gas passing through the each nostril must pass through the protruding portions, as indicated by arrows 606. Flanges 610 are provided to facilitate this sealing function so that no gas escape around the protruding portions. Flanges 610 can have any size and shape or multiple flanges can be provided to achieve this function.

In the illustrated exemplary embodiment, the outside diameter of each protruding portion increases as the distance toward base member 604 decreases. This increase in diameter is identified by numeral 612 in FIGS. 21A and 21B. The increase in diameter serves to seal the protruding portions against the nostrils to prevent gas from leaking around the patient interface.

A pair of flow element portions 614 are provided on the side of base member 604 opposite the side that protruding portions 602 are located. In an exemplary embodiment of the present invention, flow element portions 614 are integral with base member 604. A pair of rigid rings 616 are provided in flow element portions 614. Rings 616 provide a mounting structure for pressure/flow tubes 618 that are used to measuring the pressure or flow of gas passing through each channel between the patient and ambient atmosphere. More specifically, a small hole is defined in each ring and tube 618 is connected to each hole so that the flow through ring can be measured.

In the illustrated embodiment, a relatively small lip 620 is provided along an edge of each ring 616 to produce the necessary pressure drop along each channel for so that the flow or pressure can be measured. Preferably the hole in each ring for pressure or flow measurement purposes is provided adjacent to each lip because this is the location where the pressure differential in the channel is maximized.

An optional supplemental gas delivery conduit 622 is shown attached to each ring. This conduit is used, for example, to deliver oxygen to the nasal passages of the patient in the same manner conduit 508 delivers supplemental oxygen in the patient interface in the embodiment shown in FIGS. 19 and 20.

In this embodiment, holes 624 are provided on each edge of base member 604. Holes 624 help contain tubes 618 and 622 and prevent them from interfering with other medical equipment or from being distracting or annoying to the patient. Of course, the present invention contemplates any number of techniques for securing the pressure/flow sensing tube and gas delivery tubes to the patient.

Rings 616 are preferably rigid so that the cross sectional area through the rings does not change, thereby ensuring flow/pressure measuring accuracy. Because rings 616 are removable from flow element portions 614 of patient interface 600, the individual structures can be easily cleaned or components replaced as needed. It is to be understood, however, that rings 616 can be integral with flow element portions 614. Furthermore, the present invention contemplates that separate sensors can be associated with each airflow channel so that the flow of gas through each nostril can be measured independently.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A respiratory therapy and monitoring method comprising:
providing a first conduit having a proximal end, a distal end, and a first lumen defined, therethrough from the proximal end to the distal end;
delivering, via the first conduit, a flow of gas from a gas supply to an airway of a user over a plurality of respiratory cycles;
monitoring a gas flow characteristic using a pressure sensor, a flow sensor, or both operatively coupled to the airway of the user while the flow of gas is passing through the conduit;
accounting for an offset in the gas flow characteristic caused by the flow of gas; and
determining at least one respiratory variable of such a user based on the gas flow characteristic and the accounting for the offset.

2. The method of claim 1, wherein the respiratory variables include time related respiratory variables selected from the group consisting of: breathing frequency, inspiratory time, expiratory time, and inspiratory/expiratory ratio.

3. The method of claim 1, wherein the respiratory variables include:
(1) pressure related respiratory variables selected from the group consisting of: inspiratory positive airway pressure, expiratory positive airway pressure, continuous positive airway pressure, a pressure of gas in the conduit, airway pressure changes associated with user breathing, or an integral or derivative of the airway pressure changes associated with user breathing,
(2) flow related respiratory variables selected from the group consisting of: a rate of the flow of gas in the conduit, a volume of flow over a time period or over a portion of a respiratory cycle, flow associated with user breathing, or an integral or derivative of the flow associated with user breathing, or
(3) a combination of the pressure related respiratory variables and the flow related respiratory variables.

4. The method of claim 1, wherein monitoring a gas flow characteristic includes operatively coupling the pressure sensor, the flow sensor, or both the airway of the user via the first conduit.

5. The method of claim 1, wherein monitoring a gas flow characteristic includes operatively coupling the pressure sensor, the flow sensor, or both to the airway of the user via a second conduit operatively coupled to the airway of such a user.

6. The method of claim 1, further comprising administering a medicament to such a user in conjunction with delivering the flow of gas from the gas supply.

7. The method of claim 1, further comprising removing motion artifact from the gas flow characteristic.

8. The method of claim 1, wherein providing the first conduit includes inserting a bacteria filter attached to the proximal end of the first conduit at least partially into a receptacle defined in an exterior of a housing.

9. The method of claim 1, wherein monitoring a gas flow characteristic is accomplished using a flow sensor and a pressure sensor, wherein accounting for the offset includes removing a bias from an output of the flow sensor due to the flow of gas being carried by the first conduit to produce a flow signal without bias, and further comprising:
determining a zero flow point when the flow signal without bias corresponds to a substantially zero rate of flow;
determining a pressure drop in the first conduit with the pressure sensor at the zero flow point;
determining a first rate of flow for the flow of gas from the gas supply into the first conduit with the flow sensor at the zero flow point;
determining a resistance of the first conduit based on the pressure drop and the first rate of flow; and
determining a quantitative flow Q(t) for the flow of gas in the first conduit based on the resistance and an output of the pressure sensor as the respiratory variable.

10. The method of claim 1, further comprising:
determining a resistance of a user's nostril using a sizing gage; and
determining a quantitative flow Q(t) for the flow of gas in the first conduit based on the resistance determined using the sizing gage.

11. A respiratory therapy and monitoring system comprising:
a first conduit having a proximal end adapted to be coupled to a supply of gas, a distal end, and a first lumen defined, therethrough from the proximal end to the distal end;
a first sensor operatively coupled to the airway of the user while the flow of gas is passing through the first conduit, wherein the first sensor monitors a characteristic indicative of pressure or flow in the first conduit; and
processor adapted to (a) account for an offset in the characteristic caused by the flow of gas to the airway of the user, and (b) determine at least one respiratory variable of such a user based on the characteristic and the offset.

12. The system of claim 11, wherein the first sensor is in fluid communication with the first conduit.

13. The system of claim 11, wherein the respiratory variables include time related respiratory variables selected from the group consisting of: breathing frequency, inspiratory time, expiratory time, and inspiratory/expiratory ratio.

14. The system of claim 11, wherein the first sensor is a flow sensor, a pressure sensor, or both, and wherein the processor determines, as the respiratory variable, a rate of the flow of gas in the conduit, a volume of flow over a time period or over a portion of a respiratory cycle, flow associated with user breathing, or an integral or derivative of the flow associated with user breathing, inspiratory positive airway pressure, expiratory positive airway pressure, continuous positive airway pressure, a pressure of gas in the conduit, airway pressure changes associated with user breathing, an integral or derivative of the airway pressure changes associated with user breathing, or any combination thereof based on an output of the flow sensor, the pressure sensor, or both.

15. The system of claim 11, wherein the first sensor is operatively coupled to the airway of the user via the first conduit.

16. The system of claim 11, further comprising a second conduit operatively coupling the first sensor to the airway of the user.

17. The system of claim 11, further comprising means for removing motion artifact from the characteristic.

18. The system of claim 11, further comprising:
a bacteria filter disposed at the proximal end of the first conduit; and
a housing containing the first sensor, wherein the housing includes a receptacle defined in an exterior of the housing, wherein the bacteria filter and the receptacle are configured and arranged such that at least a portion of the bacteria filter is adapted to be disposed in the receptacle.

19. The system of claim 11, wherein the first sensor is a flow sensor, and further comprising a pressure sensor operatively coupled to the airway of the user, and wherein the processor (a) removes a bias from an output of the flow sensor due to the flow of gas being carried by the first conduit to produce a flow signal without bias, (b) determines a zero flow point when the flow signal without bias corresponds to a substantially zero rate of flow, (c) determines a pressure drop in the first conduit at the zero flow point; (d) determines a first rate of flow for the flow of gas from the gas supply into the first conduit at the zero flow point; (e) determines a resistance of the first conduit based on the pressure drop and the first rate of flow; and (f) determines a quantitative flow Q(t) for the flow of gas in the first conduit based on the resistance and an output of the pressure sensor as the respiratory variable.

20. The system of claim 11, further comprising a sizing gage adapted to estimate a resistance of a user's nostril, and wherein the processor determines a quantitative flow Q(t) for the flow of gas in the first conduit based on the resistance determined using the sizing gage.

21. A method of displaying a respiratory characteristic of a user, comprising:
displaying a time varying respiratory characteristic over at least a portion of a user's respiratory cycle during a current (n) respiratory cycle; and
displaying the time varying respiratory characteristic over at least a portion of such a user's respiratory cycle during a prior respiratory cycle, and wherein the time varying respiratory characteristic during the current (n) respiratory cycle and the time varying respiratory characteristic during the prior respiratory cycle are displayed in a superimposed fashion.

22. The method of claim 21, further comprising displaying the time varying respiratory characteristic over at least a portion of such a user's respiratory cycle during a plurality of prior respiratory cycles, and wherein the time varying respiratory characteristic during the current (n) respiratory cycle and the time varying respiratory characteristic during the plurality of prior respiratory cycles are displayed in a superimposed fashion.
